SEQUENCE LISTING (4 sequences)

SEQ ID NO: 1 - protein, 15 aa, SARS-CoV-2
  spike protein (1)..(15): Designed peptide based on spike protein AA sequence 326-340
IVRFPNITNLCPFGE

SEQ ID NO: 2 - protein, 15 aa, SARS-CoV-2
  spike protein (1)..(15): Designed peptide based on spike protein AA sequence 449-463
YNYLYRLFRKSNLKP

SEQ ID NO: 3 - protein, 9 aa, SARS-CoV-2
  spike protein (1)..(9): Designed peptide based on spike protein AA sequence 718-726
FTISVTTEI

SEQ ID NO: 4 - protein, 9 aa, SARS-CoV-2
  envelope protein (1)..(9): Designed peptide based on envelope protein AA sequence 2-10
YSFVSEETG

(19) United States
(12) Patent Application Publication
(10) Pub. No.: US 2025/0341521 A1
(43) Pub. Date: Nov. 6, 2025

(54) NOVEL COV-2 ANTIBODIES

(71) Applicant: R.G.C.C. Holdings AG, Zug (CH)

(72) Inventor: Ioannis PAPASOTIRIOU, Oberägeri (CH)

(73) Assignee: R.G.C.C. Holdings AG, Zug (CH)

(21) Appl. No.: 17/926,251

(22) PCT Filed: May 18, 2021
(86) PCT No.: PCT/EP2021/063134

§ 371 Date: Nov. 18, 2022

(30) Foreign Application Priority Data
May 19, 2020 (EP) 20175426.4
Jun. 19, 2020 (EP) 20181136.1

(51) Int. Cl. G01N 33/569; C07K 16/10

(52) U.S. Cl. CPC G01N 33/56983; C07K 16/1003; G01N 2333/165; G01N 2469/10

(57) ABSTRACT

The present invention provides a novel method for the production of truly fully human monoclonal antibodies against SARS-CoV-2 using isolated human blood cells. These antigens may include but are not limited to peptide sequences found in envelope or spike proteins of SARS-CoV-2 proteins.

### TECHNICAL FIELD

The present invention relates to techniques in the field of antibodies and their production. More particularly, the present invention relates to a method for producing human monoclonal antibodies that are specific for a specific antigen. This invention also relates to an antibody against SARS-CoV-2 antigens and the use of these antibodies in diagnosing, preventing and/or treating COVID-19.

### PRIOR ART

Monoclonal antibody therapies have been approved for over 30 targets and diseases with cancer being at the top. The first generation monoclonal antibodies were of murine origin and as such, unsuitable for therapeutic use due to human anti-mouse antibody response (HAMA). As recombinant DNA technology evolved, a second generation of monoclonal antibodies using chimerization or humanization was developed, thus making the antibodies more human-like. For this, genetic engineering is used to generate antibodies with human constant domains in order to decrease immunogenicity and mouse variable domains for specificity. However, humanization does not entirely and predictably preclude serious side-effect such as catastrophic system organ failures, as has been documented in the case of Theralizumab. It is thus desirable to provide fully human antibodies to avoid such side-effect arising from non-human fragments in the antibody. However, the term “fully human” can be considered misleading since antibodies, which have been designated as “fully human” have in fact a human sequence but they originate from either bacteriophages, transgenic animals or from B cell transformation. It can thus not be warranted that such antibodies are identical, in both structure and immunogenicity, to endogenously produced antibodies are. Therefore, in the context of the present invention, the term “fully human antibodies” relates to antibodies that not only have a human sequence but are also produced by human cells, just as in the in vivo process. In particular, the human cells that are employed are autologous cells to further reduce side-effects based on immunogenicity. The previously used techniques do not come without problems; and in the case of B cell transformation, the B cells producing antibodies have to be selected and then immortalized by Epstein-Barr virus (EBV) and while in vitro infection efficiency is generally high, only a small percentage of cells actually become transformed (˜1-3%). Inefficiency, instability, low yield and affinity are the characteristics of EBV transformation technology. Phage display technology involves antibody-library preparation first, followed by ligation of the variable heavy and light PCR products into a vector and finally in vitro selection of monoclonal antibody (mAb) clones. However, phage display may not recover all antigen-specific mAbs present in a given antibody library, and in vitro pairing may not reflect the in vivo process and is a complicated, demanding, and time-consuming technology. In transgenic animals, human antibody genes are inserted into for example a mouse genome, enabling human antibody production. However, the human body carries a collection of millions of antibodies and transgenic mice can only express a small fraction of this diversity. Therefore limited germline repertoire, low protein expression, residual immunogenicity and the high cost and labor involved are important drawbacks of DNA recombinant technology. However, the greatest problem of all is the simple fact that they are all essentially hybrids. There is thus a need to provide means that will enable the provision of monoclonal antibodies that can be directed against a given antigen and which are less or not immunogenic in the human body and thus limit undesirable side-effects due to immunogenicity. Of the five antibody classes, IgG is the most frequently used for cancer immunotherapy because it is a potent activator of the immune system

Previous attempts have been made for the production of human monoclonal antibodies. Fang Xu et al (2017) produced IgM human antibody by activating T cells with pulsed DCs. Then separately activated B cells with the addition of CpG, ODN or KLH and then finally combining the activated T cells with the activated B cells.

However, using CpG, ODN or ssRNA, which are human analogues of DNA microbial motifs, for cell activation and to mount a more pronounced immune response can lead to the production of antibodies against the CpG, ODNs or ssRNAs used along with the antigen of choice. Since they are normal occurring parts of human DNA, where enzymes like methyltransferases bind, the production and binding of such unwanted antibodies to their target could affect normal homeostasis. Moreover, CpG induces T-cell independent differentiation and antibodies generated this way tend to have lower affinity and are less functionally versatile.

WO 2011/023705 concerns the production of human IgG antibodies by activating T-cells with pulsed DCs, and then activating B-cells with activated T-cells. Once more, aActivation is achieved withby the use of CpG, ODN or ssRNA as well as factors like IL-12 and IFN-γ antibodies.

US 2013/0196380 concerns the production of human IgG by activating T-cells with pulsed DCs in the presence of factors like IL-4, IL-5, IL-6 and IL-10. B-cells were separately activated with the used of CpG and then added to activated T-cells.

There are also attempts to produce antibodies ex-vivo using murine cells as stated in EP 2 574 666 A1.

### SUMMARY OF THE INVENTION

Accordingly, the present invention has for its object to provide a novel process for the production of a truly fully human monoclonal antibody from isolated human blood cells, which antibody can be directed against a specific antigen of choice, thereby circumventing the above-mentioned problems that may be encountered when producing antibodies from antibody libraries through the techniques such as bacteriophages, transgenic animals or from B cell transformation. In addition, the present invention allows to broaden the range of antigens can be used in the production of a truly fully monoclonal antibodies. The antibodies obtained are not a product of genetic engineering of the cells and are not produced in a transgenic animal.

In one embodiment, an antibody against a spike protein of SARS-COV-2 is obtained, in particular against an amino acid sequence of said spike protein at positions 326-340. The amino acid sequence is a 15-mer according to SEQ ID 1. The antigen may for example be obtained by peptide synthesis techniques such as solid phase peptide synthesis. The antigen according to SEQ ID 1 has the sequence of H-IVRFPNITNLCPFGE-OH.

In one embodiment, an antibody against a spike protein of SARS-COV-2 is obtained, in particular against an amino acid sequence of the antigen at positions 449-463. The amino acid sequence is a 15-mer according to SEQ ID 2. The antigen may for example be obtained by peptide synthesis techniques such as solid phase peptide synthesis. The antigen according to SEQ ID 1 has the sequence of H-YNYLYRLFRKSNLKP-OH.

In one embodiment, an antibody against a spike protein of SARS-COV-2 is obtained, in particular against an amino acid sequence of the antigen at positions 718-726. The amino acid sequence is a 15-mer according to SEQ ID 3. The antigen may for example be obtained by peptide synthesis techniques such as solid phase peptide synthesis. The antigen according to SEQ ID 3 has the sequence of H-FTISVTTEI-OH.

In one embodiment, an antibody against a envelope protein of SARS-COV-2 is obtained, in particular against an amino acid sequence of the antigen at positions 2-10. The amino acid sequence is a 9-mer according to SEQ ID 4. The antigen may for example be obtained by peptide synthesis techniques such as solid phase peptide synthesis. The antigen according to SEQ ID 4 has the sequence of H-YSFVSEETG-OH.

In the process of the present invention, by mimicking natural mechanisms found in the adaptive immune system, dendritic, CD4+ and CD19+ cells are driven towards Th2 immunity where newly formed plasma cells then produce the antibody against the antigen of choice, depending on the antigen used. Cell activation, both dendritic, CD4+and CD19+, is succeeded by a cytokine cocktail simulating the in vivo inflammatory environment, whereas IgG production can be promoted by IgG class switching factors. The used antigens to obtain the desired antibody are peptides that were chosen for their ability to elicit immune responses. As a result of the process according to the present invention, CD138+ cells, also known as plasma or antibody producing cells, secrete anti SARS-CoV-2 antibodies, depending on the antigen used. The aforementioned plasma cells can be rendered immortal by fusion with for example a HUNS1 cell line, and were found to also produce anti SARS-CoV-2, in particular SARS-CoV-2 spike protein or envelope protein IgG antibody after immortalization.

It is therefore an object of the present invention to provide in general a process for the production of fully human, and more preferably monoclonal antibodies against a predefined antigen such as for example SARS-CoV-2, in particular against its spike and/or envelope proteins and moreover against the epitope in its spike and/or envelope proteins according to any of SEQ ID 1 to 4, said process comprising the steps of:
a) isolating peripheral blood mononuclear cells, preferably from blood;
b) generating mononuclear cells from the isolated peripheral blood mononuclear cells;
c) generating immature dendritic cells from the generated mononuclear cells;
d) isolating CD4+ and CD19+ cells, preferably from blood;
e) optionally pulsing at least the generated immature dendritic cells with the predefined antigen, such as SARS-CoV-2, in particular with its spike and/or envelope protein and moreover with the epitope in its spike and/or envelope proteins according to any of SEQ ID 1 to 4;
f) co-culturing the immature dendritic cells, the CD4+ cells and CD19+ cells;
g) pulsing at least the co-cultured immature dendritic cells, the CD4+ cells and CD19+ cells with at least the predefined antigen, such as SARS-CoV-2, in particular with its spike and/or envelope protein and moreover with the epitope in its spike and/or envelope proteins according to any of SEQ ID 1 to 4;
h) generating mature dendritic cells from the immature dendritic cells by further co-culturing the immature dendritic cells, the CD4+ cells and CD19+ cells;
i) inducing plasma cell formation
j) inducing or not inducing Ig class switching in the formed plasma cells

It is further an object of the present invention to provide an antibody against a predefined antigen, obtained in general according to the process as described above, such as for example a human antibody against SARS-CoV-2, in particular against its spike and/or envelope proteins and moreover against the epitope in its spike and/or envelope proteins according to any of SEQ ID 1 to 4.

It is further an object of the present invention to provide a human antibody against SARS-CoV-2, in particular against its spike and/or envelope proteins and moreover against the epitope in its spike and/or envelope proteins according to any of SEQ ID 1 to 4, wherein said human antibody recognizes the amino acid sequence according to SEQ ID 1 to 4 and wherein it is preferably secreted by human plasma cells, more preferably by immortalized human plasma cells, as well as its use for the determining the presence or absence of SARS-CoV-2, in particular of its spike and/or envelope proteins and moreover of the epitope in its spike and/or envelope proteins according to any of SEQ ID 1 to 4 in a sample or its use for treating COVID-19.

It is yet further an object of the present invention to provide a method for detecting the presence or absence of SARS-CoV-2, in particular of its spike and/or envelope proteins and moreover of the epitope in its spike and/or envelope proteins according to any of SEQ ID 1 to 4 in a biological sample comprising the steps of
a. contacting the sample with the above antibody, and
b. detecting the presence or absence of an antibody-antigen complex formed by the antibody and SARS-CoV-2, in particular its spike and/or envelope proteins and moreover the epitope in its spike and/or envelope proteins according to any of SEQ ID 1 to 4 in said sample.

Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1. shows the absorbance at 450 nm obtained via an ELISA test, in which antibodies generated according to the process of the invention against each of the peptides according to SEQ ID1 to 4 were tested. As can be seen, each of the antibodies led to a higher absorbance compared to the blank when contacted with the peptide it was raised against.

FIG. 2 shows a photograph of an Elispot-type assay for the evaluation of the presence of plasma cells specific for each of the SARS-COV-2 peptides according to SEQ ID 1 to 4. The first two wells from the left were incubated with inactivated B cells and show no positive color signal. The last four wells correspond to each of the peptide according to SEQ ID 1 to 4 incubated with the respective plasma cells and show a positive color signal.

### DESCRIPTION OF PREFERRED EMBODIMENTS

The process according to the present invention consists of the production of a true and fully human monoclonal antibody from human blood cells by mimicking the in vivo process.

The process according to the present invention provides in general a process for the production of preferably human, antibodies against a predefined antigen such as for example SARS-CoV-2, in particular against its spike and/or envelope proteins and moreover against the epitope in its spike and/or envelope proteins according to any of SEQ ID 1 to 4, said process comprising the steps of:
a) isolating peripheral blood mononuclear cells, preferably from blood;
b) generating mononuclear cells from the isolated peripheral blood mononuclear cells;
c) generating immature dendritic cells from the generated mononuclear cells;
d) isolating CD4+ and CD19+ cells, preferably from blood;
e) optionally pulsing at least the generated immature dendritic cells with the predefined antigen, such as SARS-CoV-2, in particular with its spike and/or envelope protein and moreover with the epitope in its spike and/or envelope proteins according to any of SEQ ID 1 to 4;
f) co-culturing the immature dendritic cells, the CD4+ cells and CD19+ cells;
g) pulsing at least the co-cultured immature dendritic cells, the CD4+ cells and CD19+ cells with at least SARS-CoV-2, in particular with its spike and/or envelope protein and moreover with the epitope of its spike and/or envelope proteins according to any of SEQ ID 1 to 4;
h) generating mature dendritic cells from the immature dendritic cells by further co-culturing the immature dendritic cells, the CD4+ cells and CD19+ cells;
i) inducing plasma cell formation
j) inducing or not inducing Ig class switching in the formed plasma cells

In the initial step of the process according to the present invention, peripheral blood mononuclear cells from a bodily sample such as for example a bodily fluid, preferably from blood. In a preferred embodiment, the peripheral blood mononuclear cells (PBMCs) can be isolated using density gradient centrifugation, for example from freshly collected blood samples in vacutainers containing EDTA. Suitable separating solutions for use in density gradient centrifugation can be obtained from VWR under the trademark BIOCOLL. After density gradient centrifugation, the cell pellet comprising peripheral blood mononuclear cells can be re-suspended in a cell culturing medium such as for example RPMI medium supplemented with 10% FBS, 200 mM L-glutamine. Cell number and viability can be determined by Trypan Blue exclusion dye.

In the subsequent step b), the thus isolated peripheral blood mononuclear cells are incubated in a cell culturing medium in order to generate mononuclear cells from the isolated peripheral blood mononuclear. In a preferred embodiment, the peripheral blood mononuclear cells were incubated in a cell culturing medium in order to generate mononuclear cells at 37° C., 5% CO₂, in particular until adherence of mononuclear cells.

In a next step c), immature dendritic cells are generated from the previously generated mononuclear cells. After the incubation period yielding adherence of mononuclear cells, the supernatant is collected and adhered mononuclear cells were washed twice with warm phosphate-buffered saline (PBS). In a preferred embodiment, the mononuclear cells were incubated in the presence of granulocyte-macrophage colony-stimulating factor (GM-CSF) and interleukin 4 (IL-4), until immature dendritic cells are generated. In order to provide optimal conditions for the generation of the immature dendritic cells from the previously generated mononuclear cells, both granulocyte-macrophage colony-stimulating factor (GM-CSF) and interleukin 4 (IL-4), were replenished together with culturing medium every 2 days. The culture was kept until it was combined with a culture of CD4+ and CD19+ cells, as obtained in step d).

In a next step d), CD4+ and CD19+ cells were isolated from a bodily sample such as for example a bodily fluid, preferably from blood. In a preferred embodiment, the CD4+ cells can be isolated using anti-human CD4 magnetic beads. After centrifugation, the pellet of anti-human CD4 magnetic beads was re-suspended in RPMI culture medium containing 10% FBS and the supernatant is kept. Cell number and viability can be determined by Trypan Blue exclusion dye.

The supernatant that was kept and anti-human CD19 magnetic beads are added to isolate CD19+ by centrifugation. After centrifugation, the pellet of anti-human CD19 magnetic beads was re-suspended in complete medium. Cell number and viability can be determined by Trypan Blue exclusion dye.

The day of the co-culture, while iDC were pulsed with the peptide such as the amino acid sequence according to the epitope in the spike and/or envelope protein according to any of SEQ ID 1 to 4, CD4+ and CD19+ cells were isolated from freshly collected whole blood samples using the same method described before. Thus, the generated immature dendritic cells from step c) or e) were combined with the isolated CD4+ and CD19+ cells from step d) into a co-culture in step f).

In an optional next step e), which may be carried simultaneously to step d), the generated immature dendritic cells are pulsed with the predefined antigen such as SARS-CoV-2, in particular with its spike and/or envelope proteins and moreover with one or more epitopes of its spike and/or envelope proteins according to any of SEQ ID 1 to 4. In a preferred embodiment, the immature dendritic cells are pulsed with the predefined peptide such as one or more epitopes of its spike and/or envelope proteins according to any of SEQ ID 1 to 4 for to a period of approximately at least 4 hours, or from 4 hours to 10 hours or for 10 hours.

In the case where an antibody against SARS-CoV-2 is to be obtained, the antigen any peptide according to SEQ ID 1 to 4, possessing the same immunogenicity as the whole spike or envelope protein, is used. By choosing a short sequence of 9 to 15 residues as antigen, the possibility of polyclonal antibody generation is decreased. The antigen can be obtained by peptide synthesis techniques such as solid phase peptide synthesis.

After generating immature dendritic cells and optionally pulsing the immature dendritic cells with the predetermined antigen such as SARS-CoV-2, in particular with its spike and/or envelope proteins and moreover with one or more epitopes of its spike and/or envelope proteins according to any of SEQ ID 1 to 4, and after isolating CD4+ and CD19+ cells, the immature dendritic cells, the CD4+ cells and CD19+ cells are combined and co-cultured in a step f). In a preferred embodiment, the ratio between immature dendritic cells to CD4+ cells and to CD19+ cell is such that the number of CD4+ cells and CD19+ cells are present in excess with respect to the number of immature dendritic cells and/or the number of CD4+ cells and CD19+ cells is approximately the same. As an example, a suitable number ratio between immature dendritic cells to CD4+ cells and to CD19+ cell is 1:10:10. In a preferred embodiment, the immature dendritic cells, the CD4+ cells and CD19+ cells are co-cultured in a suitable culturing medium, preferably RPMI culturing medium supplemented with 10% FBS, 200 mM L-glutamine and more preferably further comprising GM-CSF, IL-4, TNF-α, sCD40L, IL-6, IL-21, IL-10 and anti-human IgM. An exemplary RPMI culturing medium supplemented with 10% FBS, 200 mM L-glutamine can have 100 ng/ml GM-CSF, 50 ng/ml IL-4, 5 ng/ml TNF-α, 1 μg/ml sCD40L, 150 ng/ml IL-6, 50 ng/ml IL-21, 100 ng/ml IL-10, and 5 μg/ml IgM. A suitable culturing medium, such as for example RPMI culturing medium optionally supplemented with 10% FBS and 200 mM L-glutamine, may comprise a combination of GM-CSF, IL-4, TNF-α, sCD40L, IL-6, IL-21, IL-10 and anti-human IgM, in about 50-200 ng/ml GM-CSF, 2-100 ng/ml IL-4, 1-100 ng/ml nTNF-α, 0.5-50 μg/ml sCD40L, 50-500 ng/ml IL-6, 1-200 ng/ml IL-21, 30-300 ng/ml IL-10 and 1-50 μg/ml IgM. It is understood that where a given culturing medium is used in step f), the same culturing medium will be preferably used at least in the ensuing steps g) through j).

In a next step g), the immature dendritic cells, the CD4+ cells and CD19+ cells being co-cultured are pulsed with at least the predefined antigen such as SARS-CoV-2, in particular with its spike and/or envelope proteins and moreover with one or more epitopes of its spike and/or envelope proteins according to any of SEQ ID 1 to 4. In a preferred embodiment, the antigen is added to the RPMI culturing medium optionally supplemented with 10% FBS and 200 mM L-glutamine, and more preferably further comprising GM-CSF, IL-4, TNF-α, sCD40L, IL-6, IL-21, IL-10 and anti-human IgM, preferably at a concentration of approximately 10 μg/ml The antigen is preferably added at a concentration of approximately 10 μg/ml within the first day of co-culturing the immature dendritic cells, the CD4+ cells and CD19+ cells.

In an next step h), mature dendritic cells were generated from the immature dendritic cells, preferably by co-culturing the immature dendritic cells, the CD4+ cells and CD19+ cells until mature dendritic cells are generated in the RPMI culturing medium optionally supplemented with 10% FBS and 200 mM L-glutamine, and more preferably further comprising GM-CSF, IL-4, TNF-α, sCD40L, IL-6, IL-21, IL-10 and anti-human IgM. During co-culturing, CD4+ and CD19+ cells are activated.

In the RPMI culturing medium optionally supplemented with 10% FBS and 200 mM L-glutamine, and more preferably further comprising GM-CSF, IL-4, TNF-α, sCD40L, IL-6, IL-21, IL-10 and anti-human IgM, GM-CSF is used for maturation of immature dendritic cells, antigen processing and antigen presentation, IL-4 is used for maturation of immature dendritic cells, inhibition of macrophage development, Th2 response and high MHCII expression, TNF-α was used as an inflammatory mediator for dendritic cells and T-cell activation, Th2 differentiation, MHCII up-regulation, sCD40L was used in dendritic cells for antigen presentation, MHCII upregulation, enhanced survival, for T-cell priming and CD4 expansion and for CD19 proliferation, IL-6, an inflammatory mediator was used for lymphocyte differentiation and cell survival/proliferation, IgM mimicked BCR binding to its cognate antigen and IL-10 and IL-21 were used for IgG class switching. There are several studies indicating the role of GM-CSF, IL-4 and TNF-α on DC maturation^{i,ii}. However, a 2 stage maturation process involving GM-CSF and IL-4 also yielded immature dendritic cells.

The co-culture of immature dendritic cells, the CD4+ cells and CD19+ cells is carried out using antigen and factors mentioned below. In summary, GM-CSF was used for dendritic cells maturation, antigen processing and antigen presentation, IL-4 was used for DC maturation, inhibition of macrophage development, Th2 response and high MHCII expression, TNF-α was used as an inflammatory mediator for DC and T-cell activation, Th2 differentiation, MHCII up-regulation, sCD40L was used in DCs for antigen presentation, MHCII upregulation, enhanced survival, for T-cell priming and CD4 expansion and for CD19 proliferation, IL-6, an inflammatory mediator was used for lymphocyte differentiation and cell survival/proliferation, IgM mimicked BCR binding to its cognate antigen and IL-10 and IL-21 were used for IgG class switching

In a next step i), plasma cell formation is induced, and in particular the CD19+ cells in the co-culture of immature dendritic cells, the CD4+ cells and CD19+ cells form plasma cells. Depending on the antigen used in the process according to the present invention, plasma cells will secrete pure and fully human monoclonal antibodies against the predetermined antigen such as SARS-CoV-2, in particular its spike and/or envelope proteins and moreover against one or more epitopes of its spike and/or envelope proteins according to any of SEQ ID 1 to 4.

In a next step j), Ig class switching can be induced, and can be induced in particular in the formed plasma cells. Ig class switching allows excising unwanted Ig genes in the plasma cells so that only the desired gene can be expressed. B-cells such as CD19+ cells express IgM/IgD in their surface, but once activated can express IgA, IgE, IgG or retain their IgM expression depending on the stimuli received by T-cells. Thus in a preferred embodiment of the present invention, it can be advantageous to induce Ig class switching towards IgG expression in the plasma cells. In the RPMI culturing medium optionally supplemented with 10% FBS and 200 mM L-glutamine, and more preferably further comprising GM-CSF, IL-4, TNF-α, sCD40L, IL-6, IL-21, IL-10 and anti-human IgM, IL-10 and IL-21 were added to the co-culture to facilitate IgG class switching.

In a further step k), the plasma cells producing the antibody can be immortalized by fusion with a cancer cell line or any other technique like e.g. DNA rearrangement. In order to immortalize the plasma cells, the plasma cells generated from the co-culture can be isolated by flow cytometry using CD138-PE and then be fused to HUNSI cells. As an example, CD138 positive plasma cells can be added with approximately 10 times the number of HUNS1 cells and the fusion can be carried out using 50% PEG solution, following the protocol of the manufacturer Sigma. Finally, fused cells were let to growth in RPMI, 10% FBS, 200 mM L-glutamine until loss of IgG secretion.

In a preferred embodiment, the cells that are isolated are autologous cells, which is advantageous in particular when the antibodies are used in a therapeutical context, as the antibodies can then also be considered to be autologous.

The antibodies of the present invention may then be isolated after step j) or k) using well-known techniques in the art such as for example, but not limited to, physicochemical fractionation or affinity purification.

The antibodies of the present invention can be used to detect the presence of SARS-CoV-2, in particular its spike and/or envelope proteins and moreover one or more epitopes of its spike and/or envelope proteins according to any of SEQ ID 1 to 4 in biological samples such as for example in blood samples or mucus samples, any one of which samples may be collected via nasopharyngeal swab. The antibodies can be used to detect SARS-CoV-2 found in biological samples such as blood or mucus samples of a patient which is suspected of being impeded by or afflicted by COVID-19. Reagents and techniques for qualitatively and quantitatively determining the presence or absence of a particular antigen using an antibody are well known in the art. Examples are for example ELISA and Western blotting.

The antibodies of the present invention can thus be used to diagnose infection by SARS-CoV-2 or COVID-19 using the antibodies of the present invention. The antibodies of the present invention can be used in diagnostics because they specifically bind to spike and/or envelope protein of SARS-CoV-2 as set forth above. It has been further been found that the monoclonal antibodies of the present invention may limit the proliferation of SARS-CoV-2.

The antibodies of the present invention can be used as biomarkers in diagnose of COVID-19.

In a further embodiment of the present invention, the antibodies of the present invention can also to form antibody conjugates which can be conjugated to particles, small molecules or drugs. The antibody conjugates can be used in medical imaging and detection and/or targeted drug delivery and/or other therapeutic interventions.

The antibodies of the present invention can be used as active ingredient in pharmaceutical compositions for preventing and/or treating COVID-19.

The antibodies of the present invention can be used to prevent and/or treat COVID-19 by decreasing viral proliferation.

The present invention provides antibodies against SARS-CoV-2, in particular its spike and/or envelope proteins and moreover one or more epitopes of its spike and/or envelope proteins according to any of SEQ ID 1 to 4. Using the antibodies of the present invention it is possible to readily and reliably detect the presence or absence of SARS-CoV-2. The present invention is thus useful in the field of medical diagnosis and treatment. Further, the antibodies of the present invention can be also used in the field of pharmaceuticals such as COVID-19 diagnosis and treatment because they affect the proliferation of SARS-CoV-2.

### Experimental Data

### mAb Generation

For plasma cell generation capable of antibody production, first immature dendritic cells were generated from mononuclear cells and pulsed with the peptide of choice, i.e. one peptide selected from SEQ ID 1 to 4. Then, immature dendritic cells were fully matured in the presence of CD4 and CD19 cells. CD4 cells were activated and CD19 cells were transformed into antibody producing plasma cells. During the whole procedure, cells were incubated in the presence of growth factors mimicking inflammatory environment and promoting IgG class switch.

In order to isolate IgG antibody against any of the peptides according to SEQ ID 1 to 4 from culture supernatants, affinity chromatography was used. The collected samples were passed through a MAb Trap Kit as per manufacturer's protocol for IgG isolation. The eluent obtained contained IgG antibodies against the specific peptide used in cell culture, i.e. against any of peptides according to SEQ ID 1 to 4.

### Elisa

The antibodies produced against the 4 different peptides according to SEQ ID 1 to 4 were tested in an Elisa experiment. For each test, the bottom of the well was covered with the corresponding peptide. Supernatants containing the produced antibodies were added in each well and incubated overnight. Blank cells were incubated with medium alone as a blank comparative. After incubation, the wells were washed thoroughly and incubated with anti-human antibody conjugated with horseradish peroxidase for 3 hours. Wells were washed with phosphate buffer saline thoroughly and incubated with 3,3′,5,5′-tetramethylbenzidine (TMB) for color development. The reaction was stopped with 100 μl stop buffer. Absorbance was measured in a TECAN spectrophotometer at 450 nm. Results are shown as mean % absorbance values=SEM.

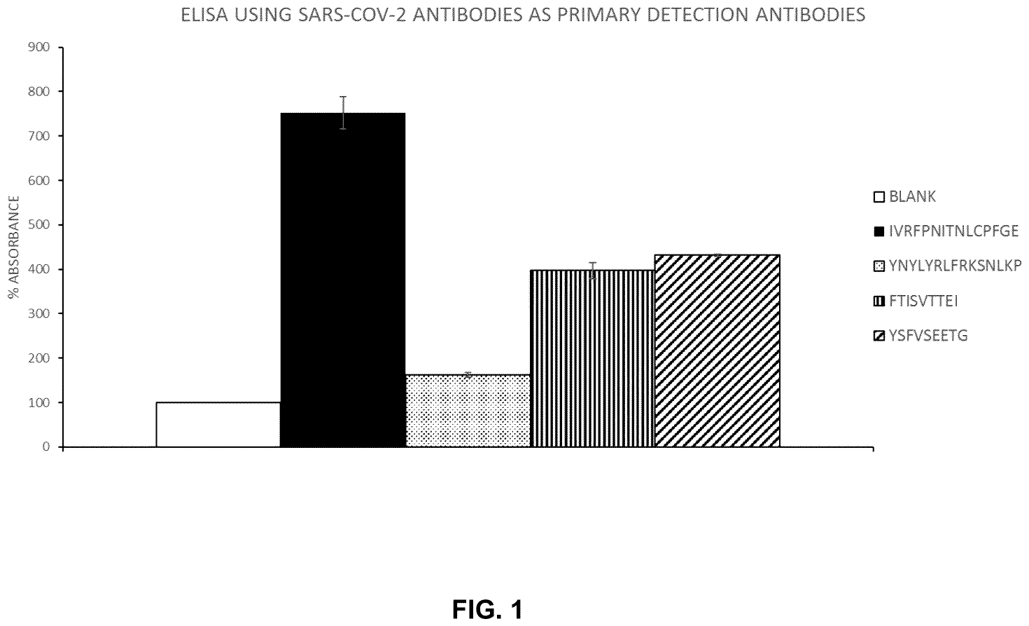

All wells incubated with the produced antibodies have significantly higher absorbance compared to blank. (blank vs Seq1 peptide p=6,5E-05; blank vs Seq2 peptide p=0,0005; blank vs Seq3 peptide p=0,0001; blank vs Seq4 peptide p=1.5E-07). Results can be seen in FIG. 1.

### Elispot

An Elispot-type assay for the evaluation of the presence of plasma cells specific for each of the SARS-COV-2 peptides according to SEQ ID 1 to 4 was carried out. Plasma cells are activated B cells against each specific SARS-COV-2 peptide according to SEQ ID 1 to 4 and display IgGs specific for said peptides in their outer membrane as well as secrete them.

96 well plates were covered with PVDF membrane and incubated with 10 μg/ml of each SARS-COV-2 peptides according to SEQ ID 1 to 4, i.e. IVRFPNITNLCPFGE, YNYLYRLFRKSNLKP, FTISVTTEI and YSFVSEETG. Plasma cells from the final co-culture were isolated and added in a PVDF containing 96 well plate that was covered with each peptide for an overnight incubation. Wells were then washed with PBS thoroughly and incubated with anti-human antibody conjugated with Alkaline Phosphatase for 3 hours. Wells were then washed thoroughly with PBS and incubated with NBT/BCIP solution.

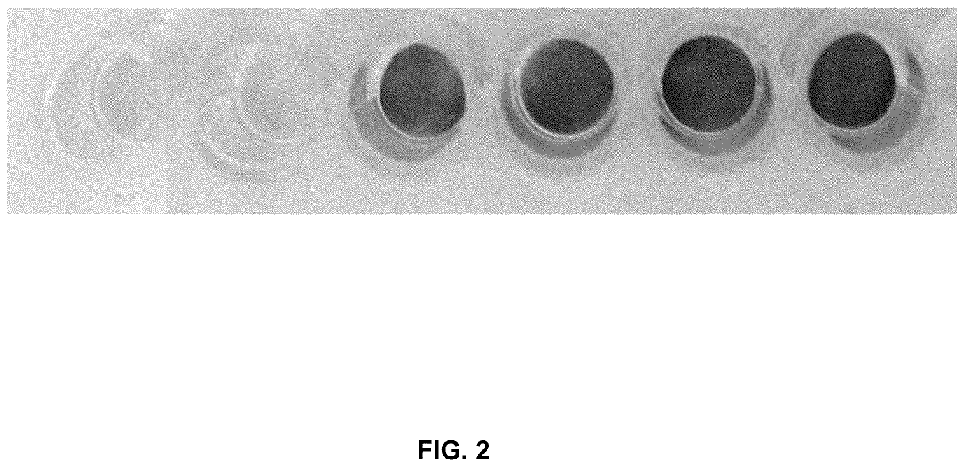

The first two wells from the left were incubated with inactivated B cells and show no positive signal for two of the peptides. The last four wells correspond to each of the peptide according to SEQ ID 1 to 4 (IVRFPNITNLCPFGE, YNYLYRLFRKSNLKP, FTISVTTEI and YSFVSEETG) incubated with the respective plasma cells. All four wells show positive signal denoting the presence of plasma cells activated against each specific peptide, as shown in FIG. 2.

1-8. (canceled)

9. A process for the production of monoclonal human antibodies by human cells, against a spike and/or envelope protein of SARS-CoV-2, said process comprising the steps of:
a) isolating peripheral blood mononuclear cells,;
b) generating mononuclear cells from the isolated peripheral blood mononuclear cells;
c) generating immature dendritic cells from the generated mononuclear cells;
d) isolating CD4+ and CD19+ cells;
e) optionally pulsing at least the generated immature dendritic cells with SARS-CoV-2, in particular with its spike and/or envelope proteins or one or more epitopes of its spike and/or envelope proteins according to any of SEQ ID 1 to 4;
f) co-culturing the immature dendritic cells, the CD4+ cells and CD19+ cells in a culturing medium comprising a combination of GM-CSF, IL-4, TNF-α, sCD40L, IL-6, IL-21, IL-10 and anti-human IgM;
g) pulsing at least the co-cultured immature dendritic cells, the CD4+ cells and CD19+ cells with at least a spike and/or envelope protein of SARS-CoV-2;
h) generating mature dendritic cells from the immature dendritic cells by further co-culturing the immature dendritic cells, the CD4+ cells and CD19+ cells;
i) inducing plasma cell formation;
j) optionally inducing or not inducing Ig class switching towards IgG expression in the formed plasma cells.

10. The process according to claim 9, where the formed plasma cells are subsequently immortalized by either by fusion with a cancer cell line or DNA rearrangement.

11. The process according to claim 9, wherein in step g) the co-cultured immature dendritic cells, the CD4+ cells and CD19+ cells are pulsed with at least a spike and/or envelope protein of SARS-CoV-2 having one or more epitopes chosen among SEQ ID 1 to 4.

12. A monoclonal antibody against a spike and/or envelope protein of SARS-CoV-2, obtained according to the process of claim 9, wherein the antibody is a human antibody produced by human cells.

13. The monoclonal antibody against a spike and/or envelope protein of SARS-CoV-2 according to claim 12, wherein the monoclonal human antibody against the spike and/or the envelope protein of SARS-CoV-2 is against one or more epitopes chosen among SEQ ID 1 to 4.

14. A monoclonal human antibody against a spike and/or envelope protein SARS-CoV-2, wherein said human antibody recognizes one of the amino acid sequences according to SEQ ID 1 to 4 and wherein it is secreted by human plasma cells.

15. A method for detecting the presence or absence of a spike and/or envelope protein of SARS-CoV-2 in a biological sample comprising the steps of
a. contacting the sample with an antibody according to claim 12, and
b. detecting the presence or absence of an antibody-antigen complex formed by the antibody and a spike and/or envelope protein of SARS-CoV-2, in said sample.

16. A method for detecting the presence or absence of a spike and/or envelope protein of SARS-CoV-2 in a biological sample comprising the steps of
a. contacting the sample with an antibody according to claim 12, and
b. detecting the presence or absence of an antibody-antigen complex formed by the antibody and a spike and/or envelope protein of SARS-CoV-2, in said sample.

17. A method of treating a human patient suffering from COVID-19 comprising the step of administering a medicament comprising an effective amount of a human antibody produced by a human cell against a spike and/or envelope protein of SARS-CoV-2, to said patient.

18. A method of treating a human patient suffering from COVID-19 comprising the step of administering a medicament comprising an effective amount of a human antibody produced by a human cell against an epitope chosen among SEQ ID 1 to 4 of a spike and/or envelope protein of SARS-CoV-2, to said patient.

19. A method for detecting the presence or absence of a spike and/or envelope protein of SARS-CoV-2 in a biological sample comprising the steps of
a. contacting the sample with an antibody according to claim 13, and
b. detecting the presence or absence of an antibody-antigen complex formed by the antibody and a spike and/or envelope protein of SARS-CoV-2, in said sample.

20. A method for detecting the presence or absence of a spike and/or envelope protein of SARS-CoV-2 in a biological sample comprising the steps of
a. contacting the sample with an antibody according to claim 13, and
b. detecting the presence or absence of an antibody-antigen complex formed by the antibody and a spike and/or envelope protein of SARS-CoV-2, in said sample.

21. A method for detecting the presence or absence of a spike and/or envelope protein of SARS-CoV-2 in a biological sample comprising the steps of
a. contacting the sample with an antibody according to claim 14, and
b. detecting the presence or absence of an antibody-antigen complex formed by the antibody and a spike and/or envelope protein of SARS-CoV-2, in said sample.

22. A method for detecting the presence or absence of a spike and/or envelope protein of SARS-CoV-2 in a biological sample comprising the steps of
a. contacting the sample with an antibody according to claim 14, and
b. detecting the presence or absence of an antibody-antigen complex formed by the antibody and a spike and/or envelope protein of SARS-CoV-2, in said sample.
